(19) United States
(12) Patent Application Publication
(10) Pub. No.: US 2021/0085474 A1
(43) Pub. Date: Mar. 25, 2021

(54) MODULAR HUMERAL STEM WITH FINE ADJUSTMENT

(71) Applicant: Tornier, Inc., Bloomington, MN (US)

(72) Inventor: Robert Courtney, JR., Pierceton, IN (US)

(21) Appl. No.: 17/044,746

(22) PCT Filed: Mar. 27, 2019
(86) PCT No.: PCT/US2019/024399

§ 371 Date: Oct. 1, 2020

Related U.S. Application Data
(60) Provisional application No. 62/652,421, filed on Apr. 4, 2018.

(51) Int. Cl. A61F 2/40

(52) U.S. Cl. CPC A61F 2/4014; A61F 2002/407; A61F 2002/4051; A61F 2002/4062

(57) ABSTRACT

A humeral stem assembly (100) includes a stem portion (104), a metaphyseal portion 108, and an intermediate spacer (112). The stem portion has a stem engagement feature (144) that has a first plurality of teeth (148). The stem engagement feature is located on a superior end of the stem portion. The metaphyseal portion has a metaphyseal engagement feature (184). The metaphyseal engagement feature can include a second plurality of teeth (188). The metaphyseal engagement feature is located on an inferior end of the metaphyseal portion. The intermediate spacer has a first spacer engagement feature (160) and a second spacer engagement feature (168). The first and second spacer engagements have a third plurality of teeth (164) on an inferior end of the intermediate spacer, and a fourth plurality of teeth (172) on a superior end of the intermediate spacer, respectively.

### BACKGROUND OF THE INVENTION

### Field of the Invention

The present application relates to apparatuses and methods for humeral implants and for the adjustment of positioning and/or orientation of the same.

### Description of the Related Art

A typical anatomical shoulder joint replacement attempts to mimic anatomic conditions. For example, a humeral stem and a humeral head replacement are attached to the humerus of the arm and replace the humeral side of a shoulder joint that is arthritic, has suffered trauma or otherwise requiring replacement to improve the condition of the patient. The humeral head replacement can articulate with the native glenoid socket or with an opposing glenoid resurfacing device.

For more severe cases, a reverse reconstruction can be performed, which includes reversing the kinematics of the shoulder joint. A reverse shoulder prosthesis can be provided by securing a semi-spherical device (sometimes called a glenosphere) to the glenoid and implanting a humeral stem with a cavity capable of receiving the glenosphere.

Before implanting the humeral implant, it may be desirable to trial the humeral implant to determine an appropriate length of the stem, appropriate inclination angle of the articulating head, and/or size of the articulating head, or other characteristics of the implant. The trial humeral implant can be assembled and then inserted into the humerus. Afterwards, the entire trial implant can be removed, and the definitive humeral implant can be chosen and implanted in the bone.

### SUMMARY OF THE INVENTION

Accordingly, there is a need for additional humeral stem assemblies that enhance a surgeon's ability to position and orient the assembly during implantation. A wider range of possible version adjustments, including finer version adjustments, can allow for increased precision in positioning the assembly and to more closely match an appropriate position for a particular patient. Such assemblies preferably include components and configurations that reduce, e.g., minimize stress risers while enhancing durability.

In one embodiment a humeral stem assembly is provided that includes a stem portion, a metaphyseal portion, and an intermediate spacer. The stem portion has a stem engagement feature that has a first plurality of teeth. The stem engagement feature is located on a superior end of the stem portion. The metaphyseal portion has a metaphyseal engagement feature. The metaphyseal engagement feature can include a second plurality of teeth. The metaphyseal engagement feature is located on an inferior end of the metaphyseal portion. The intermediate spacer has a first spacer engagement feature and a second spacer engagement feature. The first spacer engagement feature has a third plurality of teeth on an inferior end of the intermediate spacer. The second spacer engagement feature has a fourth plurality of teeth on a superior end of the intermediate spacer. The third plurality of teeth of the first spacer engagement feature is configured to engage with the first plurality of teeth of the stem engagement feature at a first interface. The fourth plurality of teeth of the second spacer engagement feature is configured to engage with the second plurality of teeth of the metaphyseal engagement feature at a second interface. A number of teeth of the third plurality of teeth differs from a number of teeth of the fourth plurality of teeth.

In another embodiment, a humeral stem assembly is provided that includes a stem and a metaphyseal portion. The stem has a first portion and a second portion. The first portion has a first end for insertion into a humerus and a second end opposite the first end. A first engagement feature is located on the second end of the first portion. The second portion has a first end that has a second engagement feature and a second end opposite the first end that has a third engagement feature. The metaphyseal portion has a fourth engagement feature. The fourth engagement feature is located on a first end of the metaphyseal portion. The metaphyseal portion is rotatable in a first direction about a longitudinal axis of the assembly to a plurality of discrete positions defined by engaging the fourth engagement feature with the third engagement feature. The second portion of the stem is rotatable in a second direction about the longitudinal axis to a plurality of discrete positions defined by engaging the second engagement feature with the first engagement feature. A version adjustment greater than zero and less than 10 degrees can be provided by a rotation of the metaphyseal portion in the first direction relative to the second portion of the stem and a rotation of the second portion of the stem in the second direction relative to the first portion of the stem.

In another embodiment a method of adjusting version of a humeral stem is provided. A metaphyseal portion, an intermediate portion, and a stem portion are assembled. The intermediate portion is slid into engagement with the stem portion at a first interface. The intermediate portion is slid into engagement with the metaphyseal portion at a second interface. Version of the humeral stem is adjusted by rotating the intermediate portion at the first interface to a discrete position in a first direction about a longitudinal axis of the implant. Version of the humeral stem is adjusted by rotating the metaphyseal portion at the second interface to a discrete position in a second direction about the longitudinal axis. The first direction is opposite the second direction. The version is adjusted by a non-zero angle less than an angular spacing between discrete positions defined at the first interface and at the second interface.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 is a schematic view of a human shoulder joint showing the bones thereof;

FIG. 2 is a schematic view of a superior humerus segment prior to resection illustrating a humeral stem assembly disposed therein;

FIG. 3 shows a humeral stem assembly applied to a humerus resected at a humeral head (in phantom lines) and having bone loss inferior to the humeral head (in sold lines);

FIG. 4 is an exploded view of a humeral stem assembly according to one embodiment;

FIG. 5 is a top view of a first portion of a humeral stem of the embodiment of FIG. 4;

FIG. 6 is a detailed side view of a portion of an engagement surface of a metaphyseal portion of the embodiment of FIG. 4;

FIG. 7 is a detailed bottom view of a portion of an engagement surface of the metaphyseal portion of the embodiment of FIG. 4;

FIG. 8 is a perspective view of an intermediate spacer according to one embodiment;

FIG. 9 is a front view of an intermediate spacer according to one embodiment;

FIG. 10 is a cross-sectional view of the intermediate spacer of FIG. 9 taken at section plane 10-10;

FIG. 11 shows a detailed side view of a bottom portion of the intermediate spacer of FIG. 9;

FIG. 12 shows a detailed side view of a top portion of the intermediate spacer of FIG. 9;

FIG. 13 is a simplified exploded view of a humeral stem assembly illustrating a method for providing version adjustment;

FIG. 14 is a detailed view of an initial version arrangement at a first and second interface; and

FIG. 15 is a detailed view of another version arrangement at a first and second interface.

### DETAILED DESCRIPTION OF THE PREFERRED EMBODIMENT

Humeral implants can include modular components. It may be desirable to be able to adjust the configuration of the assembly with a small number of modular components. For example, it would be advantageous to provide a great deal of adjustability of a biomechanical aspect of the assembled humeral implant, such as version, with a small number of components. It would be advantageous to be able to adjust version or another biomechanical aspect by small amounts, e.g., by non-zero amounts less than ten degrees. These adjustments can enable the humerus to have a proper orientation to the glenoid and/or to an articular component coupled with the glenoid.

I. Shoulder Joint Anatomy and Fine Version Adjustable Stem

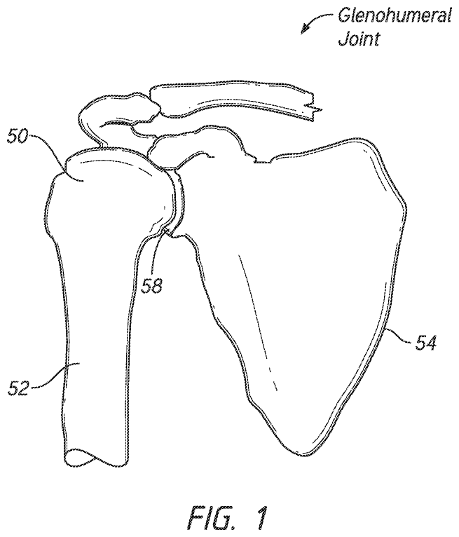

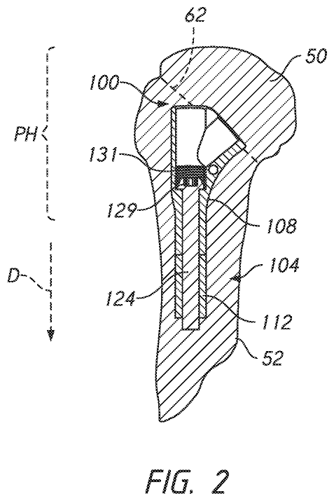

FIG. 1 shows anatomy of a glenohumeral joint. The joint is formed in part by a head 50 of a humerus 52 and a glenoid 58 of a scapula 54. The head 50 is a convex structure that is generally spherical. The glenoid 18 includes a concave articular surface upon which the head 50 moves. As discussed above, the humerus has a proximal portion that is the portion of the humerus adjacent to the glenoid 58 and forming part of the shoulder joint. The proximal humerus is sometimes referred to herein as the superior humerus. Proximal and distal in this sense are shown on FIG. 2 with reference to the humerus. In this application a location that is distal to another location refers to being closer to an inferior or elbow adjacent end of the humerus. A distal portion of the humerus is sometimes referred to herein as an inferior portion of the humerus. When the glenohumeral joint is arthritic, diseased or damaged, therapy can include forming an incision over the joint to provide access to the head 50 of the humerus 52 and the glenoid 58 of the scapula 54. Once the head 50 is accessible the head can be separated from the rest of the humerus 52 as an early part of a method of placing an implant.

FIG. 2 shows that the head 50 can be severed from the rest of the humerus 52 at the anatomical neck 62. This can be accomplished in any suitable manner, such as by directing a saw blade (not shown) along the anatomical neck 62. Once the saw blade has been passed entirely along the anatomical neck 62 the head 50 can be removed from the humerus 52 leaving an exposed resected surface. FIG. 2 shows schematically the placement of a humeral stem assembly 100 within the humerus 52. The humeral stem assembly 100 can be placed within the humerus 52 such that a stem 104 can be embedded within the distal humerus D. A portion of a metaphyseal portion 108 is left accessible at the resection surface made at the anatomic neck 62.

As discussed further below, in some cases the humerus 52 may be fractured or otherwise damaged by trauma or disease such that a portion of the humerus 52 including the head 50 and a portion distal to the anatomic neck 62 has been removed or is not present. In such cases one portion of the stem 104 can be embedded in the remaining portion of the humerus 52 and another portion can extend proximally from the embedded portion.

The following embodiments illustrate how a fine degree of version adjustment can be provided in a stem for the humerus 52. These embodiments can be used in other orthopedic assemblies involving stems and similar structures where rotational orientation adjustment is useful.

II. Humeral Stem Assemblies

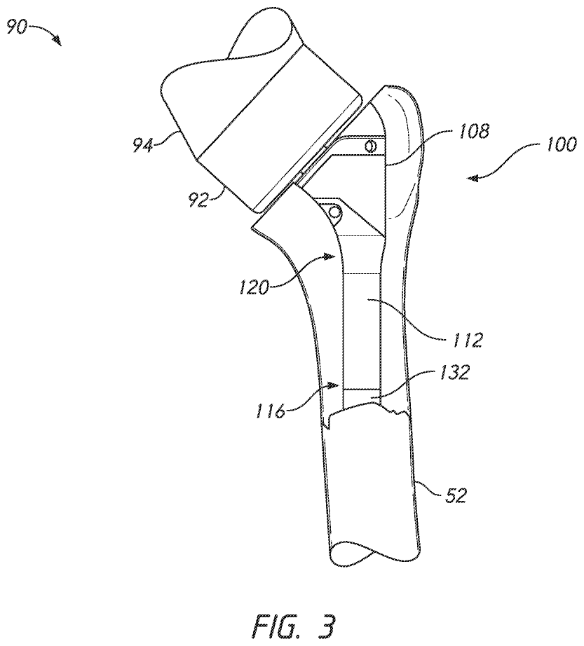

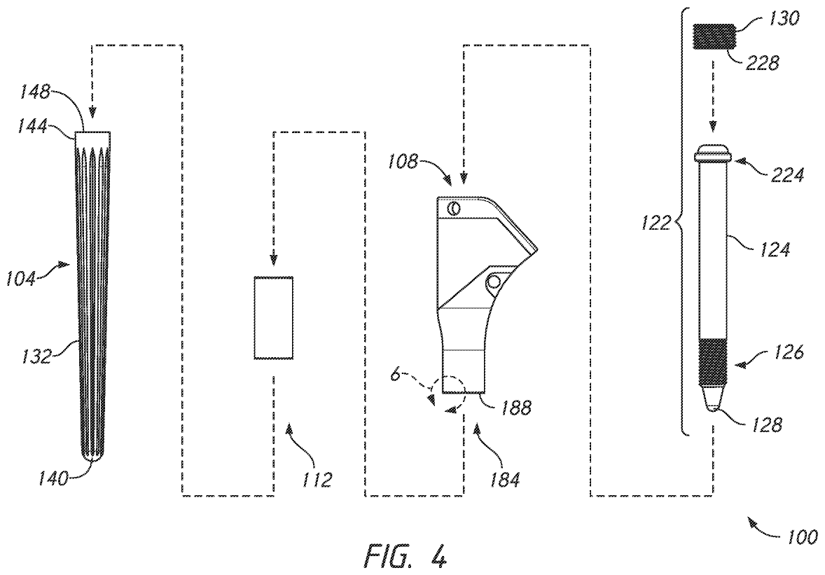

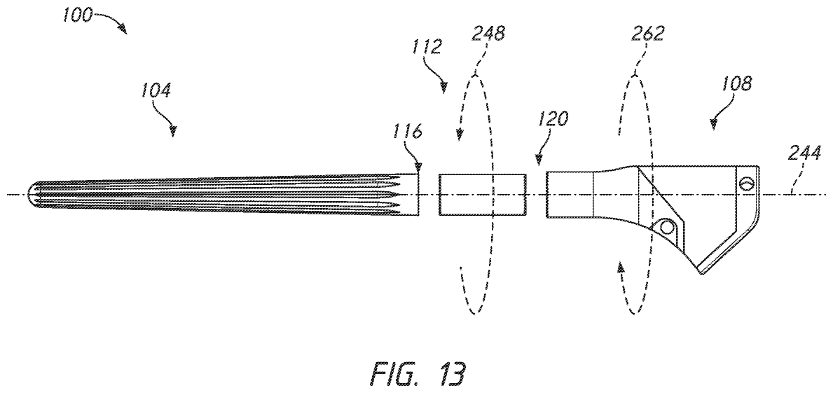

FIG. 2 shows that the humeral stem assembly 100 includes the stem 104 and the metaphyseal portion 108 as discussed above. The stem 104 can have a first portion 132 (see FIG. 4) and, in some cases, an intermediate spacer 112. The intermediate spacer 112 can be considered a second portion of the stem 104. The intermediate spacer 112 can be mated to the first portion 132 at a first interface 116 (see FIGS. 3 and 13). The intermediate spacer 112 can be mated to the metaphyseal portion 108 at a second interface 120 (see FIGS. 3 and 13). One, two or all three of the first portion 132, the intermediate spacer 112, and the metaphyseal portion 108 can be configured for internal compression connection.

For example, each of the first portion 132, the humerus 12 and the metaphyseal portion 108 can include a lumen that extends therethrough. The lumens can be aligned such that a bolt member 124 can be advanced through the metaphyseal portion 108, into the intermediate spacer 112, and further into the first portion 132. As discussed further below, the bolt member 124 can be configured to be threaded into the first portion 132. The tooling interface 129 can enable a powered or manual driver to engage a proximal end of the bolt member 124 to advance threads at a distal end thereof into thread disposed in a distal portion of the humeral stem assembly 100, e.g., within a lumen 133 of the first portion 132.

FIG. 2 shows that the humeral stem assembly 100 can be configured to be secured against inadvertent disengagement of the bolt member 124 from the other components of the humeral stem assembly 100. In one embodiment the metaphyseal portion 108 includes internal threads 131 configured to mate with a cap screw 130, discussed below in connection with FIG. 4.

FIG. 3 shows repair of a more complex humeral condition, such as a severe fracture. The humerus 52 has been modified by trauma or surgical technique such that not only is the head 50 removed but also a proximal portion including the anatomic neck 62 and a portion of the shaft of the humerus 52 has been removed. The humeral stem assembly 100 is embedded in the remaining portion of the humerus 52. The first portion 132 of the stem 104 is advanced into the cancellous bone of the humerus 52 along a central portion such as along a central portion of a humeral intramedullary canal.

The first portion 132 is coupled with the intermediate spacer 112. The intermediate spacer 112 is coupled with the metaphyseal portion 108. The first portion 132, intermediate spacer 112, and metaphyseal portion 108 are secured together in a suitable manner such as by securing the bolt member 124 in the central lumens thereof using the cap screw 130 or otherwise.

After the humeral stem assembly 100 has been assembled in the humerus 52 adjustments to biomechanical configuration of the humeral stem assembly 100 can be made. Such adjustments can include a version adjustment. In some cases, a fine version adjustment can be provided. Fine and other version adjustments are discussed below in connection with FIGS. 13-15.

After the humeral stem assembly 100 has been assembled and adjusted to the satisfaction of the surgeon a humeral assembly 90 can be provided by coupling a tray 92 with the humeral stem assembly 100. The tray 92 can include a taper on a distal side thereof and a concave recess on a proximal side thereof. The taper can be configured to be inserted into a recess formed in a proximal face of the metaphyseal portion 108. The taper and the recess can have corresponding conical configurations that facilitate an interference fit, such as by a Morse taper connection. The concave recess of the tray 92 can be configured to receive a mating projection of an insert 94 to be mated with the tray 92. The insert 94 is configured such that the mating projection is on a distal side thereof. The insert 94 can have a concave articular surface formed in a proximal side thereof. The concave articular surface of the insert 94 can be configured to articulate over a convex member configured to be coupled with the glenoid 58 of the scapula 54. The convex member is sometimes referred to as a glenosphere. The foregoing description and as depicted in FIG. 3 are illustrative of a reverse shoulder assembly. Further description of reverse shoulder configurations are described in U.S. Pat. No. 9,498,344 and in US2015/0265411A1, both of which are incorporated by reference herein in their entirety.

Although a reverse shoulder configuration is illustrated in FIG. 3 the humeral stem assembly 100 can also be used in connection with an anatomic configuration. For instance an anatomic humeral head can be provided. The anatomical humeral head comprises a convex articular surface that is configured to articulate over a concave surface, such as the native glenoid in the case of a hemi-arthroplasty or a glenoid liner member with a glenoid implant including a concave glenoid articular surface. In some example, the anatomic humeral head includes a taper on a distal side thereof, opposite the convex articular surface thereof. The taper on the distal side is configured to be inserted into a recess in the metaphyseal portion 108, which can be tapered. The connection between the anatomic humeral head and the metaphyseal portion 108 can be by a Morse taper configuration.

FIG. 4 shows more details of the humeral stem assembly 100. As discussed above the first portion 132 of the stem 104 can be inserted into a distal portion of the humerus 52. The first portion 132 can include a first end 140. The first end 140 can be the distal-most portion of the first portion 132. A second end 144 of the first portion 132 opposite the first end 140 can be the proximal-most end of the first portion 132 of the stem 104. The first portion 132 can be configured to mate with cancellous bone of the humerus 52. For example, the first portion 132 can include a plurality of channels, ridges, flutes, pores or other structures the enhance engagement with bone. These and other structures can enhance ingrowth of bone into these structures as a way to reduce, minimize or eliminate rotation or proximal motion of the first portion 132 relative to the cancellous bone matter in the humerus 52 where the first portion 132 is positioned.

The second end 144 can include a first plurality of teeth 148. The first plurality of teeth 148 can be disposed at the proximal end of the first portion 132 of the stem 104. The first plurality of teeth 148 can be disposed to be proximally facing to enable the first portion 132 to mate with the intermediate spacer 112 as indicated by the arrow extending from the intermediate spacer 112 to the second end 144 of the first portion 132. The first plurality of teeth 148 can take any suitable configuration, as discussed further below. In some embodiments the first plurality of teeth 148 has planar edges that meet at linear edges. In some embodiments the first plurality of teeth 148 includes curved faces that meet at smooth curved peaks. Linear edge peaks provide excellent mating. Smooth curved peaks provide less stress concentration compared to that of the linear edge teeth.

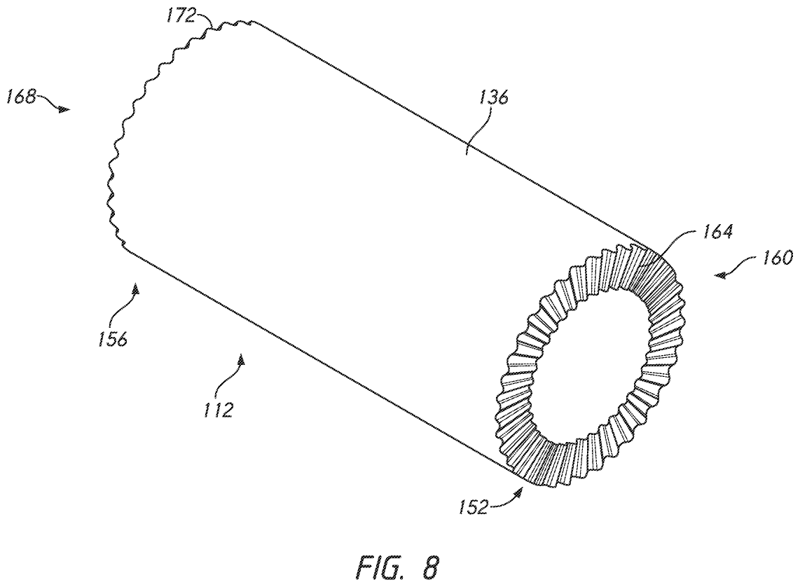

FIGS. 4 and 8 show the intermediate spacer 112 in more detail. In one embodiment, the intermediate spacer 112 forms at least a portion of a second portion 136 of the stem 104. The second portion 136 can include a cylindrical body with a lumen therethrough. The body can extend between a first end 152 and a second end 156 of the intermediate spacer 112. A first spacer engagement feature 160 can be formed or provided at the first end 152. The first spacer engagement feature 160 can include a second plurality of teeth 164. As discussed elsewhere herein the teeth of the second plurality of teeth 164 can be formed with linear edges or with rounded peaks. In one embodiment there are a number of teeth in the second plurality of teeth 164 that matches the number of teeth in first plurality of teeth 148. For example, there can be 36 teeth disposed on an end face of the body of the intermediate spacer 112 at the first end 152. The teeth of the various pluralities of teeth can be disposed on mating surfaces at the interfaces, as described herein. There can be 36 teeth disposed about the periphery of the first portion 132 of the stem 104 at the second end 144 thereof. There can also be more or less teeth in the first plurality of teeth 148 and the second plurality of teeth 164. For example, there can be about 20-35 teeth (e.g., there can be 22 teeth, 25 teeth, 30 teeth, 33 teeth, etc.). In some embodiments, the teeth of the first plurality of teeth 148 can be spaced apart by a set angle 149, such as ten degrees. In other embodiments, a different angular spacing can be provided between the teeth of the first plurality of teeth 148 and the second plurality of teeth 164. For example, there can be an angular spacing of about 10 to 18 degrees (e.g., there can be angular spacing of 12 degrees, 14.4 degrees, etc.) between teeth on the intermediate spacer 112 and on the first portion 132 of the first end 140.

The intermediate spacer 112 has a second spacer engagement feature 168 disposed at the second end 156 of the cylindrical body thereof. The second spacer engagement feature 168 can have a third plurality of teeth 172. The third plurality of teeth 172 can be disposed on a proximal facing side of the intermediate spacer 112. The third plurality of teeth 172 can have sharp or rounded configurations as discussed herein. The teeth of the third plurality of teeth 172 will be of a number different from the number of teeth of the second plurality of teeth 164. The spacing between the third plurality of teeth 172 is a different spacing than the spacing of the second plurality of teeth 164 in some embodiments. In one example the third plurality of teeth 172 includes thirty teeth and the second plurality of teeth 164 includes thirty-six teeth. In one example, the teeth of the third plurality of teeth 172 are spaced apart by twelve degrees and the teeth of the second plurality of teeth 164 are spaced apart by ten degrees. There can also be more or less teeth in the third plurality of teeth 172. For example, there can be about 20-35 teeth (e.g., there can be 22 teeth, 25 teeth, 30 teeth, 33 teeth, etc.). In other embodiments, a different angular spacing can be provided between the teeth of the third plurality of teeth 172. For example, there can be an angular spacing of about 10 to 18 degrees (e.g., there can be angular spacing of 12 degrees, 14.4 degrees, etc.) between teeth on the second spacer engagement feature 168 of the intermediate spacer 112.

FIG. 4 shows, by a dashed arrow, that the metaphyseal portion 108 can be coupled with the intermediate spacer 112 when the humeral stem assembly 100 is implanted. The metaphyseal portion 108 includes a metaphyseal engagement feature 184 at a first end. The metaphyseal engagement feature 184 can be disposed at a distal end of the metaphyseal portion 108. The metaphyseal engagement feature 184 can be disposed on the metaphyseal portion 108 such that the metaphyseal engagement feature 184 can be placed directly on the second spacer engagement feature 168 disposed at the second end 156 of the intermediate spacer 112. The metaphyseal engagement feature 184 can include a fourth plurality of teeth 188. The fourth plurality of teeth 188 can be configured to mate with the third plurality of teeth 172 disposed on the second end 156 of the intermediate spacer 112.

The teeth of the metaphyseal engagement feature 184 can be of a different number of teeth than is the teeth of the first plurality of teeth 148. The teeth of the metaphyseal engagement feature 184 can be spaced apart by a different angular separation than are the teeth of the first plurality of teeth 148. As a result, the metaphyseal portion 108 can be rotated in a first direction relative to the stem 104 or to the intermediate spacer 112 by an angular amount that is different in magnitude from a relative rotation of the first portion 132 of the stem 104 relative to the intermediate spacer 112 or another portion (e.g., a proximal portion) of the humeral stem assembly 100.

The number of teeth at the first interface 116 can be different from the number of teeth at the second interface 120. Similarly, the angular spacing between adjacent teeth at the first interface 116 can be different from the angular spacing between adjacent teeth at the second interface 120. The difference between the angular spacing between adjacent teeth at one of the two interfaces and the angular spacing between adjacent teeth at another interface can be between 0 and 10 degrees (e.g., the difference in angular spacing can be 0.5 degrees, 2 degrees, 5 degrees, etc.).

The metaphyseal portion 108 has a proximal portion that is connectable to an articular component or assembly as discussed above.

A rigid connection can be made between the stem 104, the metaphyseal portion 108, and the intermediate spacer 112 by any suitable manner For example, a connector 122 can be provided that secures these components together. The connector 122 can be in the form of an assembly that provides for secure connections. The connector 122 can include a bolt member 124 with a threaded end 126. The threaded end 126 can include threads and also a taper 128. The bolt member 124 can include a tapered head 224 at the proximal end thereof. The bolt member 124 can include a tooling interface 129 (see FIG. 2), as discussed above, for advancing the bolt member 124 into threaded connection with the first portion 132 or another portion of the humeral stem assembly 100. The connector 122 can include a cap screw 130 that secures the bolt member 124 within the humeral stem assembly 100. The cap screw 130 can include a tapered recess 228 that receives the tapered head 224 as discussed further below.

FIGS. 2 and 4 illustrate how the secure connection of the components of the humeral stem assembly 100 can be achieved. The first portion 132 can be placed within a humeral bone at a selected position, e.g., at least partially embedded therein. The intermediate spacer 112 can be placed on the first portion 132 such that the first spacer engagement feature 160 at the first end 152 engages a corresponding engagement feature at the second end 144 of the first portion 132, e.g., teeth of the second plurality of teeth 164 can be engaged with teeth of the first plurality of teeth 148 at the second end 144. Thereafter the metaphyseal portion 108 can be engaged with the intermediate spacer 112. For example the metaphyseal engagement feature 184 can be engaged with the second spacer engagement feature 168 at the second end 156 of the intermediate spacer 112. Teeth of the fourth plurality of teeth 188 can be engaged with teeth of the third plurality of teeth 172. After the first portion 132, the intermediate spacer 112, and the metaphyseal portion 108 have been engaged in this manner, the taper 128 of the bolt member 124 can be inserted into an aperture at the proximal end of the metaphyseal portion 108. The proximal end aperture can be aligned with lumens through the metaphyseal portion 108, the intermediate spacer 112 and the first portion 132 of the stem 104 respectively. The bolt member 124 can continue to be advanced into the humeral stem assembly 100 through these lumens until threads at the threaded end 126 engage corresponding threads 127 within the first portion 132 of the stem 104. This threaded engagement connects the first portion 132 of the stem 104, the intermediate spacer 112, and the metaphyseal portion 108 together. Thereafter the cap screw 130 can be advanced into the aperture at the proximal end of the metaphyseal portion 108 aligned with the lumens of the metaphyseal portion 108, the intermediate spacer 112, and the first portion 132 of the stem 104. The cap screw 130 can be engaged with threads in the metaphyseal portion 108 (see FIG. 2) and advanced along these threads until the cap screw 130 receives in the tapered recess 228 thereof the tapered head 224 of the bolt member 124. Further advancement of the cap screw 130 creates additional compression in the bolt member 124 and therefore at a first interface 116 between the first portion 132 of the stem 104 and the intermediate spacer 112 and also between at a second interface 120 between the intermediate spacer 112 and the metaphyseal portion 108.

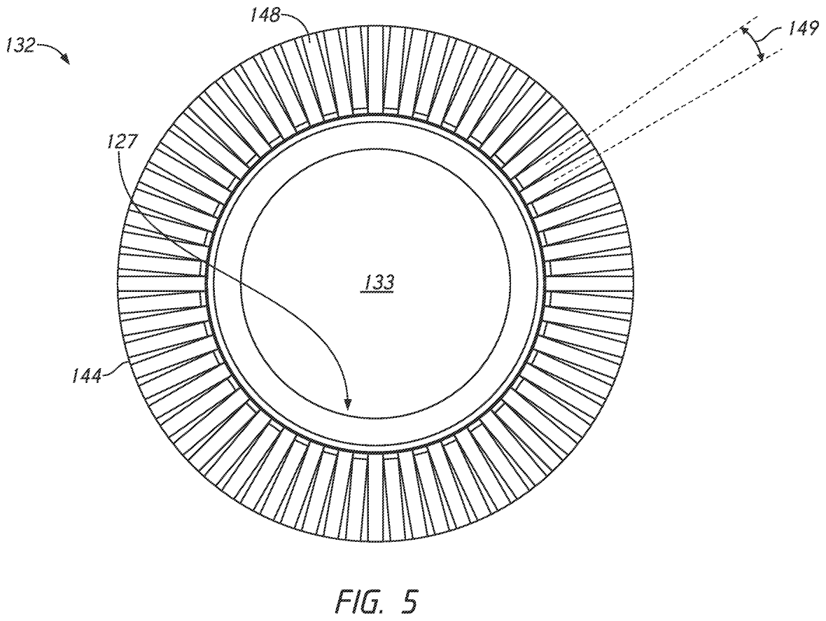

FIG. 5 shows additional features of first plurality of teeth 148 of the humeral stem assembly 100. The teeth of the first plurality of teeth 148 are disposed about the periphery of the first portion 132 on a proximal-facing surface at the second end 144 of the first portion 132. The first plurality of teeth 148 can be disposed about a peripheral annulus disposed outward of the lumen 133 on a proximal annular surface of the first portion 132. The teeth of the first plurality of teeth 148 can have any of the configurations disclosed herein including having sharp edges or smooth rounded edges as discussed further below.

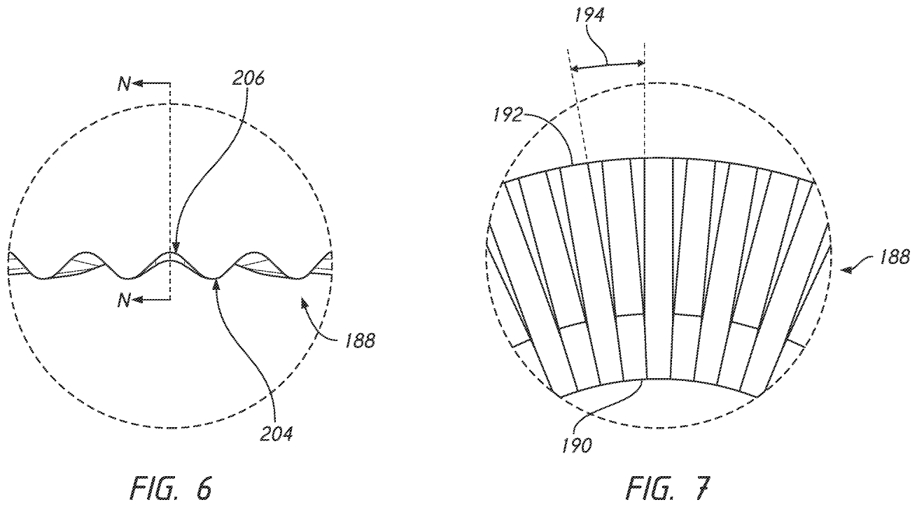

FIGS. 4, 6, and 7 show configurations of the metaphyseal portion 108 at a distal (or inferior) position thereof. As discussed above, the metaphyseal portion 108 includes the metaphyseal engagement feature 184 which can include teeth, e.g., the fourth plurality of teeth 188. FIG. 6 shows in a side view that the teeth of the fourth plurality of teeth 188 can include an arcuate configuration. The teeth can be formed as a series of peaks and troughs. The teeth can be disposed along a sinusoidal curve at the distal (or inferior) side of the metaphyseal portion 108. In some embodiments, the teeth of the fourth plurality of teeth 188 comprises a peak 204. The rounded peak 204 can be present as a series of peaks. The rounded peak 204 can be disposed between adjacent troughs 206. The number of rounded peaks 204 and rounded trough 206 can be any of those described above. FIG. 7 is a bottom view of the metaphyseal portion 108 that shows that the teeth of the fourth plurality of teeth 188 can extend from an inner periphery 190 to an outer periphery 192. The teeth can be spaced apart by an angle 194, which can be any of the angles described above.

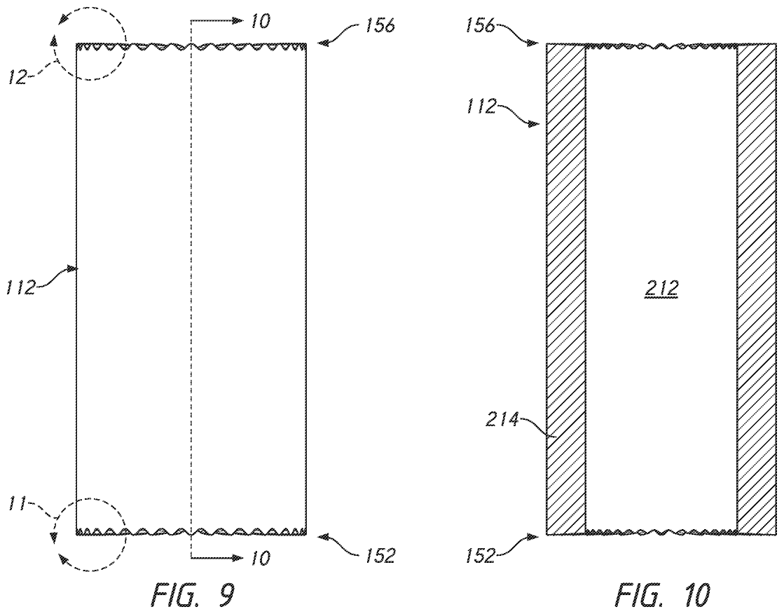

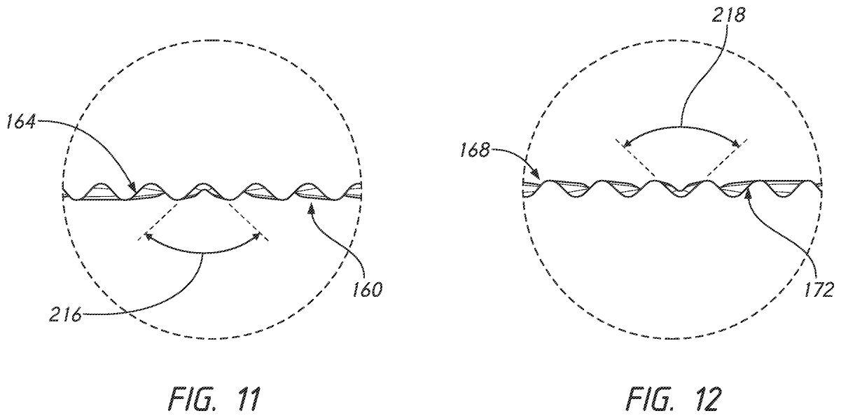

FIGS. 8-12 illustrate various aspects of the intermediate spacer 112 which has been described above. The intermediate spacer 112 includes a lumen 212 that extends between the first end 152 and the second end 156. The lumen 212 provides access for the connector 122, in particular for the shaft of the bolt member 124. The lumen 212 is enclosed within an annular wall 214 that surrounds the lumen 212 along the length of the cylindrical body 138 of the intermediate spacer 112. FIG. 11 shows a configuration of the first spacer engagement feature 160 at the first end 152. As discussed above, the first spacer engagement feature 160 can include teeth, e.g., a second plurality of teeth 164. The teeth of the second plurality of teeth 164 can be rounded as discussed above in connection with other interface. The teeth of the second plurality of teeth 164 can be of any number. The teeth of the second plurality of teeth 164 can be spaced apart at any suitable angular spacing. The teeth of the second plurality of teeth 164 can have an internal tooth angle 216 as shown in FIG. 11. The tooth angle can be measured between lines tangent to the curve forming the teeth 164. In another way, the teeth 164 can be characterized as being formed by a continuous curve that has a depth or amplitude that can be constant about the periphery of the spacer 112. The depth can be less than 1 mm, e.g., less than 0.5 mm, e.g., about 0.2 mm. The teeth 164 can have a lesser depth adjacent to the lumen 212, e.g., from about 10% to 50% to less depth than adjacent to the radial outer surface of the spacer 112, e.g., about 0.1 mm adjacent to the lumen 212. The tooth angle 216 can be any suitable angle to provide good mating with the teeth of the first plurality of teeth 148. FIG. 12 shows that the second spacer engagement feature 168 can include teeth in the third plurality of teeth 172. The teeth of the third plurality of teeth 172 can be of any number discussed above, e.g., can be the same or a different number of teeth than are in the second plurality of teeth 164. The teeth of the third plurality of teeth 172 can have a different configuration such as having a second tooth angle 218 that is different from the first tooth angle 216. The second tooth angle 218 can be a function of the number of teeth in the third plurality of teeth 172.

III. Methods of Fine Adjustment

Although it has been known to use teeth to secure components of a humeral assembly together such prior approaches did not enable fine biomechanical configuration adjustment. FIG. 13 illustrates a configuration and method for providing fine adjustment of version.

FIG. 13 is a simplified exploded view of the humeral stem assembly 100. The first portion 132 of the stem 104, the intermediate spacer 112, and the metaphyseal portion 108 are aligned along a longitudinal axis 244. The longitudinal axis 244 can be an axis aligned with the longitudinal axis of the bolt member 124 of the connector 122 (see FIG. 4). As discussed above the first portion 132 can be advanced into a humerus or a portion thereof (see FIG. 3). When the first portion 132 is secured, the position of the intermediate spacer 112 can be set. The intermediate spacer 112 can be slid into engagement with the first portion 132 at an early stage of assembly and/or adjusting version of the humeral stem assembly 100.

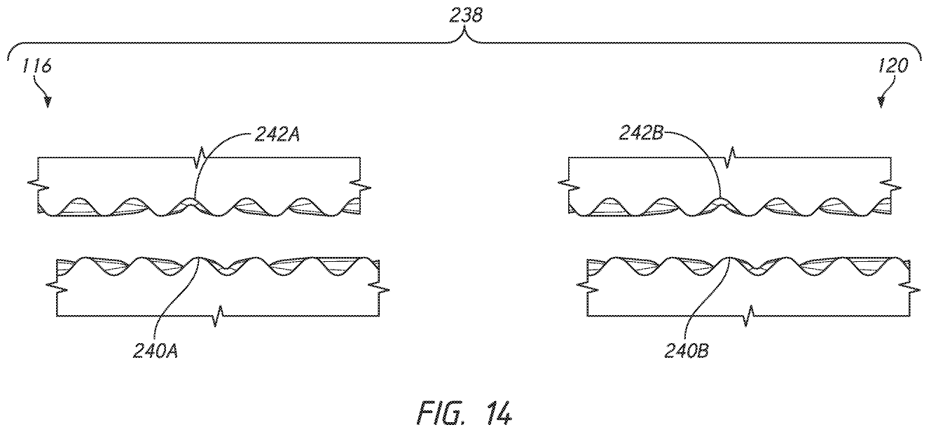

FIG. 14 illustrates an initial version 238 arrangement that can be provided by aligning an initial crest 240A of the first plurality of teeth 148 with an initial trough 242A of the second plurality of teeth 164 at the first interface 116. The initial version 238 also can include setting the second interface 120 by aligning an initial crest 240B with an initial trough 242B at the second interface 120. FIG. 14 shows a stage in which the teeth are spaced apart. This is the condition just before sliding the intermediate spacer 112 into engagement with the first portion 132 and just before sliding the metaphyseal portion 108 into engagement with the intermediate spacer 112. These steps of sliding can occur sequentially, e.g., with the intermediate spacer 112 mating with the first portion 132 at the first interface 116 initially and then with the metaphyseal portion 108 mating with the intermediate spacer 112 subsequently. The initial version 238 can correspond to a neutral position without any adjusted version. The initial version 238 can be an initial version position off-set from neutral based on a pre- or intra-operative analysis of the patient.

After the initial version is confirmed the connector 122 can be used to secure the metaphyseal portion 108, the first portion 132 of the stem 104 and the intermediate spacer 112 together. Or if the version provided by the initial version 238 is not suitable an adjustment can be made.

In one embodiment, the mating teeth at the first interface 116 are offset by 10 degrees, e.g., by providing 36 equally spaced teeth at the first interface 116. The mating teeth at the second interface 120 are offset by 12 degrees, e.g., by providing 30 equally spaced teeth at the second interface 120. Thus, an anterior or posterior adjustment of 10 degrees can be provided by rotating the intermediate spacer 112 relative to the first portion 132 of the stem 104 by one tooth but not changing the relative positions of the metaphyseal portion 108 and the intermediate spacer 112. An anterior or posterior adjustment of 12 degrees can be provided by rotating the metaphyseal portion 108 relative to the intermediate spacer 112 by one tooth but not changing the relative positions of the intermediate spacer 112 relative to the first portion 132 of the stem 104. An adjustment of 20 or 24 degrees can be provided by adjusting in the same manner by two teeth.

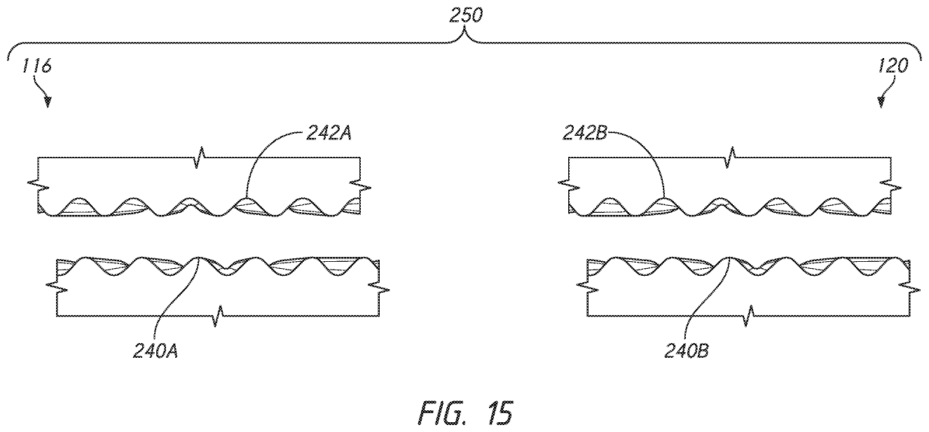

Smaller, fine adjustments can be provided by the method illustrated by FIG. 15. The intermediate spacer 112 can be rotated relative to the first portion 132 of the stem 104 in a first direction 248 about the longitudinal axis 244 (see FIG. 13). This rotation moves the initial trough 242A by one tooth relative to the initial crest 240A. The metaphyseal portion 108 can be rotated relative to the intermediate spacer 112 in a second direction 262 about the longitudinal axis 244 (see FIG. 13). This moves the initial trough 242B at the second interface 120 one tooth relative to the initial crest 240B in a direction opposite of the direction of movement of the teeth at the first interface 116. The motion at the first interface 116 causes a 10 degree rotation of the version angle and the motion at the second interface 120 causes a 12 degree rotation of the version angle in the opposite direction for a net adjustment of 2 degrees. A 4 degree adjustment can be provided by the same relative rotations by rotating by two teeth at first interface 116 and in the opposite direction by two teeth at the second interface 120. In this manner, any multiple of two degrees of adjustment can be provided by the humeral stem assembly 100.

In another embodiment, the mating teeth at the first interface 116 are offset by 14.4 degrees, e.g., by providing 25 equally spaced teeth at the first interface 116. The mating teeth at the second interface 120 are offset by 15 degrees, e.g., by providing 24 equally spaced teeth at the second interface 120. Larger or finer adjustment can be made by rotating one or both interfaces as discussed above. This configuration can provide 0.6 degree adjustment (or multiples thereof).

In another example, the teeth at the first interface 116 are spaced apart by 15 degrees and the teeth at the second interface 120 are spaced apart by 10 degrees. This enables adjustments to be made in the same manner described above in connection with FIGS. 13-15 in multiples of 5 degrees. For example, rotation of the intermediate spacer 112 relative to the first portion 132 of the stem 104 in a first direction 248 about the longitudinal axis 244 by one tooth causes a 15 degree rotation of the version angle and rotation of the metaphyseal portion 108 relative to the intermediate spacer 112 in a second direction 262 opposite the first direction 248 by one tooth causes a 10 degree rotation of the version angle in the opposite direction. As a result, the motion at the first interface 116 and at the second interface 120 causes a net adjustment of the version angle of 5 degrees.

In another example, the teeth at the first interface 116 are spaced apart by 15 degrees and the teeth at the second interface 120 are spaced apart by 14.4 degrees. This enables adjustments to be made in the same manner described above in connection with FIGS. 13-15 in multiples of 0.6 degrees. For example, rotation of the intermediate spacer 112 relative to the first portion 132 of the stem 104 in a first direction 248 about the longitudinal axis 244 by one tooth causes a 15 degree rotation of the version angle and rotation of the metaphyseal portion 108 relative to the intermediate spacer 112 in a second direction 262 opposite the first direction 248 by one tooth causes a 14.4 degree rotation of the version angle in the opposite direction. As a result, the motion at the first interface 116 and at the second interface 120 causes a net adjustment of the version angle of 0.6 degrees. A 1.2 degree adjustment can be provided by the same relative rotations but by rotating by two teeth at the first interface 116 and in the opposite direction by two teeth at the second interface 120.

Certain embodiments have been described herein with a single spacer. However, any number of spacers can be used to form the humeral implant (e.g., two, three, four, or more). Further, the spacers in the humeral implant do not need to be identical. For example, some of the spacers can have the engagement features describe above while other spacers can have different engagement features (e.g., threadable engagements, slideable engagements, etc.). As another example, the spacer can include one or more springs, stoppers, and flange portions on pins. More details of alternative spacers are disclosed in U.S. Pat. No. 9,597,203, which is hereby incorporated by reference in its entirety.

The foregoing embodiments have described humeral implant applications. These embodiments can have other orthopedic applications, including implantation in other long bones or other long bone areas, e.g., the proximal or distal femur in hip or knee procedures, distal humerus in an elbow procedure, proximal or distal radius or ulna in elbow or wrist procedures, proximal or distal tibia or fibula in knee or ankle procedures. In each of these cases, the embodiments can enable gross or fine adjustment of a component, such as a first articular surface relative to an opposing component, such as a second native or prosthetic articular surface. These embodiments can be used in other joint procedures where the orientation of the components could benefit from gross or fine adjustment, such as in spinal body fusion or spinal motion preserving implants in spinal joints.

Terminology

Certain methods are described as sliding a first component relative to a second component. However, it should be understood that relative motion between first and second components can also include sliding the second component relative to the first component. For example, “sliding the intermediate portion in a transverse direction relative to a longitudinal axis of the metaphyseal portion” can also include “sliding the metaphyseal portion in a transverse direction relative to a longitudinal axis of the intermediate portion.”

“Implant” is a broad term and is to be given its ordinary and customary meaning to a person of ordinary skill in the art and includes, without limitation, temporary implants (e.g., for trialing) or permanent implants (also referred to herein as definitive implants) for any anatomy, including, but not limited to, shoulder replacements, knew replacements, femoral replacements, and hip replacements.

As used herein, the term “metaphyseal portion” refers to either a reverse metaphyseal portion or an anatomic metaphyseal portion unless otherwise specified.

As used herein, the relative terms “proximal” and “distal” shall be defined from the perspective of the humeral component. Thus, proximal refers to the direction of the metaphyseal portion and distal refers to the direction of the stem portion.

For expository purposes, the term “transverse” as used herein is defined as a direction generally perpendicular to the longitudinal axis of the assembly, unless otherwise specified.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” “generally,” and “substantially” as used herein represent an amount close to the stated amount that still performs a desired function or achieves a desired result. For example, the terms “approximately,” “about,” “generally,” and “substantially” may refer to an amount that is within less than 10% of the stated amount, as the context may dictate.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about four” includes “four”

Any methods disclosed herein need not be performed in the order recited. The methods disclosed herein include certain actions taken by a practitioner; however, they can also include any third-party instruction of those actions, either expressly or by implication. For example, actions such as “distally moving a locking element” include “instructing distal movement of the locking element.”

Although certain embodiments and examples have been described herein, it will be understood by those skilled in the art that many aspects of the humeral assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. A humeral stem assembly comprising:
a stem portion comprising a stem engagement feature comprising a first plurality of teeth, wherein the stem engagement feature is located on a superior end of the stem portion;
a metaphyseal portion comprising a metaphyseal engagement feature comprising a second plurality of teeth, wherein the metaphyseal engagement feature is located on an inferior end of the metaphyseal portion;
an intermediate spacer comprising a first spacer engagement feature comprising a third plurality of teeth on an inferior end of the intermediate spacer and a second spacer engagement feature comprising a fourth plurality of teeth on a superior end of the intermediate spacer;
wherein the third plurality of teeth of the first spacer engagement feature are configured to engage with the first plurality of teeth of the stem engagement feature at a first interface, and wherein the fourth plurality of teeth of the second spacer engagement feature are configured to engage with the second plurality of teeth of the metaphyseal engagement feature at a second interface; and
wherein a number of teeth of the third plurality of teeth is different than a number of teeth of the fourth plurality of teeth.

2. The humeral stem assembly of claim 1, wherein the intermediate spacer comprises a cylindrical body.

3. The humeral stem assembly of claim 1, wherein the teeth of the second spacer engagement feature and the teeth of the metaphyseal engagement feature comprise a rounded peak configuration.

4. The humeral stem assembly of claim 1, wherein the teeth of the first spacer engagement feature and the teeth of the stem engagement feature comprise a rounded peak configuration.

5. The humeral stem assembly of claim 1, wherein the teeth of the second spacer engagement feature and the teeth of the metaphyseal engagement feature comprise 36 teeth.

6. The humeral stem assembly of claim 5, wherein the teeth of the first spacer engagement feature and the teeth of the stem engagement feature comprise 30 teeth.

7. The humeral stem assembly of claim 1, wherein the teeth of each of the second spacer engagement feature, metaphyseal engagement feature, first spacer engagement feature, and stem engagement feature are formed on annular surfaces.

8. The humeral stem assembly of claim 1, further comprising a lumen extending through the stem, the metaphyseal portion, and the intermediate spacer, and a connector disposed through the lumen and configured to secure the teeth at the first and second interfaces in any of a plurality of discrete positions.

9. A humeral stem assembly comprising:
a stem comprising:
a first portion having a first end for insertion into a humerus, a second end opposite the first end, a first engagement feature located on the second end of the first portion;
a second portion comprising a first end having a second engagement feature and a second end opposite the first end having a third engagement feature;
a metaphyseal portion comprising a fourth engagement feature, wherein the fourth engagement feature is located on a first end of the metaphyseal portion;
wherein the metaphyseal portion is rotatable in a first direction about a longitudinal axis of the assembly to a plurality of discrete positions defined by engaging the fourth engagement feature with the third engagement feature at a first interface and wherein the second portion of the stem is rotatable in a second direction about the longitudinal axis to a plurality of discrete positions defined by engaging the second engagement feature with the first engagement feature at a second interface; and
wherein a version adjustment greater than zero and less than 10 degrees can be provided by a rotation of the metaphyseal portion in the first direction relative to the second portion of the stem and a rotation of the second portion of the stem in the second direction relative to the first portion of the stem.

10. The humeral stem assembly of claim 9, wherein a version adjustment of about 2 degrees can be provided by rotating the metaphyseal portion relative to the second portion of the stem in the first direction by one tooth and rotating the second portion of the stem relative to the first portion of the stem in the second direction opposite the first direction of the stem by one tooth.

11. The humeral stem assembly of claim 9, wherein the first interface comprises a first angular spacing between discrete positions and the second interface comprises a second angular spacing between discrete positions.

12. The humeral stem assembly of claim 11, wherein the first angular spacing is 10 degrees and the second angular spacing is 12 degrees.

13. The humeral stem assembly of claim 11, wherein the difference between the second angular spacing and the first angular spacing is 2 degrees.

14. The humeral stem assembly of claim 11, wherein the difference between the second angular spacing and the first angular spacing is less than 1 degree.

15. A method of adjusting version of a humeral stem, the method comprising:
assembling a metaphyseal portion, an intermediate portion, and a stem portion, the assembling comprising:
sliding the intermediate portion into engagement with the stem portion at a first interface; and
sliding the intermediate portion into engagement with the metaphyseal portion at a second interface; and
adjusting version of the humeral stem by rotating the intermediate portion at the first interface to a discrete position in a first direction about a longitudinal axis of the implant and rotating the metaphyseal portion at the second interface to a discrete position in a second direction about the longitudinal axis, wherein the first direction is opposite the second direction, and wherein the version is adjusted by a non-zero angle less than an angular spacing between discrete positions defined at the first interface and at the second interface.

16. The method of claim 15, further comprising positioning the stem portion in a fractured humerus, at least a proximal end of the intermediate portion exposed.

17. The method of claim 15, wherein a distal end of the stem portion is distal a fracture line, a proximal end of the intermediate portion coupled with a distal end of the metaphyseal portion exposed.

18. The method of claim 15, further comprising rotating the intermediate portion by one tooth in the first direction and rotating the metaphyseal portion by one tooth in the second direction.

19. The method of claim 15, further comprising rotating the intermediate portion by at least two teeth in the first direction and rotating the metaphyseal portion by at least two teeth in the second direction.

20. The method of claim 15, further comprising advancing a coupler through a lumen in the metaphyseal portion, the intermediate portion, and the stem portion.
